(19) United States
(12) Patent Application Publication
(10) Pub. No.: US 2012/0294827 A1
(43) Pub. Date: Nov. 22, 2012

(54) POLYMER GEL FORMULATION

(76) Inventors: Abraham J. Domb, Efrat (IL); Boris Vaisman, Jerusalem (IL); Lior Yankelson, Tel Aviv (IL); Moran Yaniv, Azaria (IL)

(21) Appl. No.: 13/575,930

(22) PCT Filed: Jan. 28, 2011
(86) PCT No.: PCT/IB11/50384

§ 371 Date: Jul. 27, 2012

Related U.S. Application Data
(60) Provisional application No. 61/298,924, filed on Jan. 28, 2010.

(51) Int. Cl. A61K 31/765; A61K 8/85; A61Q 19/08; A61P 19/02; A61P 13/00; A61P 1/04; A61P 27/02; A61K 8/84; A61P 17/02

(52) U.S. Cl. 424/78.37

(57) ABSTRACT

A polymer gel, in at least some embodiments, featuring both cross-linked castor oil and branched castor oil components, in which the castor oil is optionally replaced by ricinoleic acid.

### FIELD OF THE INVENTION

The present invention is of a polymer gel formulation, and in particular, of a polymer gel formulation featuring both branched and cross-linked polymers.

### BACKGROUND OF THE INVENTION

Polymeric materials that are pasty or liquid are designated as gels. Various materials have been used for such gels, such as gels that are based upon hydroxy fatty acids, acrylic acid polymer, cellulose derivatives, chitosan and many others.

Cross-linking of castor oil and/or its components to form a gel has been described. For example, U.S. Pat. No. 5,387,658 describes cross-linking of castor oil and/or its derivatives, such as ricinoleic acid for example, to form a gel that is useful as a liquid thickener or emulsifier for example for lubricants, cosmetics or food; however this patent does not relate to branched polymers in combination with cross-linked polymers, and medical applications, such as soft tissue repair or augmentation, are not discussed.

### SUMMARY OF THE INVENTION

The background art does not teach or suggest a composition that forms a gel with a combination of cross-linked and branched components, and that is biodegradable, safe as an implant and is useful for medical use, soft tissue repair and augmentation.

The present invention overcomes the limitations of the background art by providing a polymer gel that, in at least some embodiments, features both cross-linked and branched polymers, which is preferably prepared with one or more of castor oil, ricinoleic acid and/or hydroxy stearic acid as a base. Although the description below centers around castor oil, ricinoleic acid may also optionally be used for all compositions and methods described below, and is understood to be encompassed therein. As described in greater detail below, the polymer gel comprises a plurality of components: at least a first component comprising cross-linked castor oil and at least a second component comprising branched castor oil. The branched castor oil may also optionally comprise a polyester, which is preferably added after the branched castor oil component is prepared.

The cross-linked material preferably comprises a cross-linker, which may optionally comprise any acid containing three or more carboxylic or alcohol groups, including but not limited to one or more of citric acid, mucic acid, tartaric acid, or a combination thereof, in an amount suitable to induce cross-linking. Preferably, the cross-linker comprises citric acid and/or mucic acid. It should be noted that by “cross-linker” it is meant any molecule capable of inducing any type of covalent bond between three other molecules. Crosslinking can be affected by a molecule having three or more alcohol or carboxylic acid groups which include castor oil, pentaerythritol, sugar molecules having three or more alcohol groups such as mannitol, glucose and sucrose, mucic acid, tartaric acid and nitrilotriacetic acid. However, carboxylic acids having two or more carboxylic acid groups are required to allow ester group formation.

In the case of castor oil, optionally the cross-linker comprises any acid containing two or more carboxylic groups, including but not limited one or more of the above cross-linkers and/or sebacic acid or succinic acid, or any combination thereof.

The amount of citric acid is preferably in an amount of at least 7% w/w with regard to castor oil, more preferably in an amount of from about 7% to about 20%, and most preferably in an amount of from about 7.5% to about 10% w/w.

The branched castor oil preferably comprises a branching agent, which may optionally comprise citric acid in an amount suitable to induce branching. The amount of citric acid is preferably in an amount of at least 0.1% w/w with regard to castor oil, more preferably in an amount of from about 0.1% to about 7%, and most preferably in an amount of from about 4% to about 7%.

The branched castor oil optionally comprises a polyester of chains of hydroxy acids such as ricinoleic acid, lactic acid, glycolic acid, and hydroxycaproic acid. The polyester is preferably also added after the branched castor oil is prepared, more preferably through reaction with a lactone, although optionally the reaction is performed through direct condensation with the hydroxy acid. The lactone may optionally comprise any type of caprolactone and/or lactide, for example D-lactide, L-lactide, or DL-lactide, or epsilon caprolactone, or a combination thereof. The polyester is optionally added through ring opening polymerization, for example through reaction with lactide or with some other suitable lactones, using zinc lactide as a catalyst or through direct condensation as previously described.

Once the cross-linked component and the branched component have been prepared, they are preferably mixed to form the polymer gel. For example, the components may optionally be mixed as powders, or one component may optionally be in powder form while the other is in paste or liquid form. Preferably, the cross-linked component is prepared as a powder, more preferably as a milled powder, and is mixed with the branched component which is preferably prepared as a liquid or paste.

Unexpectedly, the present inventors have found that the polymer gel has many useful properties. It is preferably already in its gel state before being placed in the body, so the gel composition may be sculpted, adjusted and otherwise used to volumetrically fill any void and/or to augment any soft tissue, for example.

Although, as noted above, U.S. Pat. No. 5,387,658 describes cross-linking of castor oil, the cross-linking agents are used in much greater amounts. Also the castor oil is described as being preferably partially or completely hydrogenated, while the castor oil as used herein is preferably not hydrogenated. Furthermore, this patent fails to teach or suggest the combination of cross-linked castor oil with branched castor oil; this combination was found to be surprisingly effective to achieve a suitable gel for implants, medical use, soft tissue replacement and/or augmentation. Furthermore and again without wishing to be limited in any way, this combination provides stability and persistence of the desired effects over the time, even years after implantation; it is biocompatible and provides the “feel” of natural fat when the tissue is touched externally (on the skin) in the vicinity of the implanted material.

According to at least some embodiments of the present invention and without wishing to be limited in any way, optionally the gel compositions described herein may be prepared with a simple melt condensation method with no added solvents.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows the injection locations for the animals in groups 1-4 in example 3;

FIG. 2 shows the injection locations for the animals in group 5 in example 3;

FIG. 3 shows implant weight (dry and wet) after one and three months;

FIG. 4 shows average rat weight following subcutaneous implantation;

FIG. 5 shows implant weight % after one, three and six months;

FIGS. 6-7 are photographs of tissue after 6-month evaluation (samples 5, 9, 12, 14); and

FIGS. 8-9 are photographs of tissue after 6-month evaluation (samples 5, 8, 13, 17).

### DESCRIPTION OF THE PREFERRED EMBODIMENTS

The present invention, in at least some embodiments, provides a polymer gel featuring both cross-linked and branched polymers, which is preferably prepared with one or more of castor oil, ricinoleic acid and/or hydroxy stearic acid as a base and a trifunctional molecule having at least two carboxylic acid groups.

Optionally a variety of different fatty polymer gels can be created and optimized by combining two types of polymers prepared with one or more of castor oil, ricinoleic acid and/or hydroxy stearic acid as a base, were one polymer is branched and the other polymer is cross linked in different levels of cross linking and branching and by mixing the two types of polymer to form a gel.

According to at least some embodiments, castor oil may optionally be fully or partially replaced by ricinoleic acid or hydroxy stearic acid.

In another embodiment a variety of different fatty polymer gels can be created and optimized by combining two types of polymers prepared from castor oil as a base, in which one castor oil based polymer is branched and the other castor oil based polymer is cross linked at different levels of cross linking and branching; the two types of polymer are mixed to form a gel.

According to at least some embodiments, the branched polymer, such as branched castor oil for example and without limitation, may optionally comprise a polyester of chains of hydroxy acids such as ricinoleic acid, lactic acid, glycolic acid, and hydroxycaproic acid bound to the castor oil or ricinoleic acid base.

According to at least some embodiments of the present invention and without wishing to be limited in any way, optionally the gel compositions described herein may be prepared with a simple melt condensation method with no added solvents.

Each of these materials and optional embodiments are described in greater detail below as exemplary illustrations only and without wishing to be limited in any way. Section headings are provided for the purpose of clarity only and without wishing to be limited in any way.

### Cross-Linked Polymer

The cross-linked polymer preferably comprises a cross-linker, which may optionally comprise any acid containing three or more carboxylic or alcohol groups, including but not limited to one or more of citric acid, mucic acid, tartaric acid, or a combination thereof, in an amount suitable to induce cross-linking. Preferably, the cross-linker comprises citric acid and/or mucic acid. It should be noted that by “cross-linker” it is meant any molecule capable of inducing any type of covalent bond between three other molecules. Crosslinking can be affected by a molecule having three or more alcohol or carboxylic acid groups which include castor oil, pentaerythritol, sugar molecules having three or more alcohol groups such as mannitol, glucose and sucrose, mucic acid, tartaric acid and nitrilotriacetic acid. However, carboxylic acids having two or more carboxylic acid groups are required to allow ester group formation.

In the case of castor oil, optionally the cross-linker comprises any acid containing two or more carboxylic groups, including but not limited one or more of the above cross-linkers and/or sebacic acid or succinic acid, or any combination thereof.

The amount of citric acid is preferably in an amount of at least 7% w/w with regard to castor oil, more preferably in an amount of from about 7% to about 20%, and most preferably in an amount of from about 7.5% to about 10% w/w.

Preparation of the cross-linked polymer may optionally be performed as follows (described with regard to castor oil for the purpose of illustration only and without any intention of being limiting in any way). Castor oil and 7.5% w/w citric acid are inserted into a flask with a magnetic stirrer. Optionally and preferably up to 10% citric acid is used; however as the citric acid ratio increases, the cross-linked polymer becomes stiffer, and tends to swell less or even not to swell at all. The reaction is stirred in a nitrogen atmosphere at a temperature that is at least the acid melting point temperature (130-155° C. for citric acid) until a homogenous solution is observed. After the solid acid melts, the nitrogen is removed and the reaction continues under vacuum for a suitable period of time, optionally from two days to seven days, until the liquid becomes elastomeric. Once the liquid becomes elastomeric, the magnetic stirrer or other stiffing mechanism inside it stops rotating or rotates more slowly, as once cross-linking occurs, the liquid ceases to flow or flows to a significantly reduced degree.

Then the crossed linked polymer is transferred into a suitable container, such as a flat glass pot for example, and placed in a vacuum oven. A non-limiting example of a suitable temperature and pressure is 140° C., 5-25 mbar; preferably the temperature and pressure are maintained for a suitable period of time, optionally for a period of a number of hours for example.

### Branched Polymer

The branched polymer preferably comprises a branching agent, which may optionally comprise citric acid in an amount suitable to induce branching. The amount of citric acid is preferably in an amount of at least 0.1% w/w with regard to castor oil, more preferably in an amount of from about 0.1% to about 7%, and most preferably in an amount of from about 4% to about 7%.

According to at least some embodiments, the branched polymer, such as branched castor oil for example and without limitation, may optionally comprise a polyester of chains of hydroxy acids such as ricinoleic acid, lactic acid, glycolic acid, and hydroxycaproic acid. Such a polyester is preferably also added after the branched polymer is prepared, more preferably through reaction with a lactone, although optionally the reaction is performed through direct condensation with the hydroxy acid. The lactone may optionally comprise any type of caprolactone and/or lactide, for example D-lactide, L-lactide, or DL-lactide, or epsilon caprolactone, or a combination thereof. The polyester is optionally added through ring opening polymerization, for example through reaction with lactide or with some other suitable lactones, using zinc lactide as a catalyst or through direct condensation.

Exemplary methods of preparing branched polymers, with and without polyester chains of hydroxy acids, are now described.

The following method may optionally be used to prepare branched polymer without polyester chains of hydroxy acids.

Castor oil and 4-7% w/w citric acid are inserted into a flask with stiffing, for example by a magnetic stirrer. As the amount of citric acid is increased, the polymer becomes more viscous.

The reaction is stirred in a nitrogen atmosphere at the acid melting point temperature (155° C. for citric acid; however the previously described temperature ranges for cross-linking may optionally be used) until a homogenous solution is observed. After the solid acid is dissolved, the nitrogen is removed and the reaction continues in vacuum for a suitable period of time such as 3 days. This reaction is monitored by a suitable method such as GPC (gel permeation chromatography) with specified and standardized molecular weight standards as is known in the art, and the reaction may be deemed complete and stopped when the molecular weight of the resultant branched product remains constant.

The following method may optionally be used to prepare branched polymer with polyester chains of hydroxy acids.

Addition of the polyester to the branched polymer may optionally be performed by ring opening polymerization (ROP) of lactones (caprolactone and/or lactide) on the branched polymer. The ROP occurs in the presence of a catalyst (Zn-lactide), added for example in an amount 0.1% mole per lactone. The lactones, branched polymer and the catalyst are inserted into a flask with a magnetic stirrer. The reaction is stirred in an argon atmosphere at 140° C. for 3 days. The reaction is monitored by GPC and the reaction is deemed complete and stopped when the molecular weight remains constant. Optionally caprolactone may be used in the range of from 25 to 50% w/w.

### Polymer Gel Compositions and Methods of Preparation Thereof

Once the cross-linked component and the branched component have been prepared, they are preferably mixed to form the polymer gel. For example, the components may optionally be mixed as powders, or one component may optionally be in powder form while the other is in solution. Preferably, the cross-linked component is prepared as a powder, more preferably as a milled powder, and is mixed with the branched component which is preferably prepared as a liquid or paste.

The branched polymer may also optionally comprise a polyester, which is preferably added after the branched polymer component is prepared as described above.

Before mixing, the crosslinked polymer component is prepared by milling, more preferably in liquid nitrogen, and is then sieved, for example by 10-20 mesh sieves. The resulting milled crosslinked polymer component is added to a flask with the branched polymer which is liquid. More preferably, the crosslinked polymer component is between 10% to 50% of the total mass. The mixture is stirred at a suitable temperature under nitrogen. The temperature selected preferably allows swelling and interpenetration of the components, in order to provide more effective gel formation. The resulting mixture is then allowed to cool back to room temperature in a nitrogen atmosphere. The polymer gel is then ready to be administered, for example by being inserted into syringes for injection.

The resultant gel composition preferably has a viscosity in the range of about 10⁵-10⁶ cP units (at a low shear rate) depending on the ratio of hydroxy acid monomer and castor oil and on the molecular weight of the hydroxy acid chains: as the chain length increases, the viscosity increases.

According to at least some embodiments, there is provided a method of manufacture with the gel composition through one or more of extrusion, injection and compression molding as well as particulate leaching and solvent casting.

### Cosmetic and Medical Therapeutic Uses

In at least some embodiments, the present invention relates to cosmetic and medical polymer-based moldable gel or gel-like compositions, for cosmetic and medical therapeutic uses, including but not limited to reconstruction. For such applications and without wishing to be limited in any way, the composition advantageously permits in situ formation of a custom, contoured filler or implant without invasive surgical intervention or general anesthesia.

Hereinafter, the term “treatment” includes both pretreatment, before a pathological condition has arisen, and treatment after the condition has arisen. The term “treating” includes both treating the subject after the pathological condition has arisen, and preventing the development of the pathological condition.

According to at least some embodiments, the gel composition may optionally feature controlled biomedical degradation characteristics, for biomedical applications.

According to at least some embodiments, the gel composition of the present invention may be used as part of tissue engineering and drug delivery therapies by tailoring the composition to optimize physical properties of the gel, including but not limited to viscosity, mechanical strength, elasticity and/or rate of biodegradation.

According to at least some embodiments, there is provided a gel composition for use in the chemical, food, cosmetic, or pharmaceutical industry as stabilizers or thickeners.

In an embodiment the present invention provides a composition and method of use thereof for aesthetic applications for the face including but not limited to one or more of smoothing (filling) nasolabial folds, enhancing cheekbones, augmenting lips, smoothing (filling) mentolabial folds, enhancing the chin and/or enhancing the bridge of the nose.

In an embodiment the present invention provides a composition as urethral bulking agent for the treatment of female urinary incontinence.

In an embodiment the present invention provides a composition for an injectable homogeneous polymer resulting in a unique combination of elasticity, viscosity and stability, for example (and without limitation) for intra-articular injections.

The gel compositions can be used for a variety of medical uses, implants and soft tissue repair and augmentation procedures in a subject, whether for treatment or prevention, preferably a mammal, such as humans, dogs, cats, horses, pigs, cows, and sheep. For example, the gel compositions can be used in facial tissue repair or augmentation, including, but not limited to, camouflaging scars, filling depressions, smoothing out irregularity, correcting asymmetry in facial hemiatrophy, second bronchial arch syndrome, facial lipodystrophy and camouflaging age-related wrinkles as well as augmenting facial eminences (lips, brow, etc.). Additionally, the gel compositions can be used to restore or improve sphincter function such as for treating stress urinary incontinence. Other uses of the gel compositions may also include the treatment of vesicoureteral reflux (incomplete function of the inlet of the ureter in children) by subureteric injection and the application of these gel compositions as general purpose fillers in the human body.

Surgical applications for the gel compositions include, but are not limited to, facial contouring (e.g., frown or glabellar line, acne scars, cheek depressions, vertical or perioral lip lines, marionette lines or oral commissures, worry or forehead lines, crow's feet or periorbital lines, deep smile lines or nasolabial folds, smile lines, facial scars, lips and the like); periurethral injection including injection into the submucosa of the urethra, along the urethra, and at or around the urethral-bladder junction to the external sphincter; ureteral injection for the prevention of urinary reflux; injection into the tissues of the gastrointestinal tract for the bulking of tissue to prevent reflux; to aid in sphincter muscle coaptation, internal or external, and for coaptation of an enlarged lumen; intraocular injection for the replacement of vitreous fluid or maintenance of intraocular pressure for retinal detachment; injection into anatomical ducts to temporarily plug the outlet to prevent reflux or infection propagation; larynx rehabilitation after surgery or atrophy; and any other soft tissue which can be augmented for cosmetic or therapeutic effect.

Another non-limiting exemplary application relates to injection to the joints for managing Osteoarthritis.

The concentration of the above described gel in the final administered composition can be readily determined by the attending physician based on the indication; height, weight, and/or age of the patient; and the period of time the material needs to be in place. The concentration of the polymer(s) in the composition is optionally from about 20% to about 100% by weight of the composition.

According to at least some embodiments, the gel composition may optionally be used for surgical sutures and resorbable implants, drug encapsulation and drug delivery applications (the latter are described in greater detail below).

In order to be used in medical devices and controlled-drug-release applications, also as described in greater detail below, the gel composition is biocompatible (by using biocompatible polymers, cross-linkers and branching agents) and also preferably meets other criteria to be qualified as biomaterial-processable, sterilizable, and capable of controlled stability or degradation in response to biological conditions.

### Drug Delivery Compositions

In at least some embodiments, the present invention relates to drug delivery formulations that contain biodegradable polymers and bioactive agents and methods for making these formulations. The polymer gel composition may be utilized to form medical devices, drug delivery devices, or coatings for other medical devices. The drug delivery composition may also optionally be applied for any of the therapeutic and/or cosmetic applications described above.

In at least some embodiments the gel described herein may further comprise one or more therapeutic agents, prophylactic agents, diagnostic agents, and combinations thereof. Suitable classes of active agents include, but are not limited to, anti-inflammatory agents; local anesthetics; analgesics; antibiotics; anti cancer agent, growth factors and agents that induce and/or enhance growth of tissue within the filled cavity or control the growth of a certain type of tissue, such as certain types of collagen, and combinations thereof.

Exemplary local anesthetics include, but are not limited to, lidocaine and bupivacaine. Exemplary antiinflammatory agents include, but are not limited to, triamcinolone, dexamethasone, ibuprofen, and indomethacin. Exemplary antibiotics include, but are not limited to, gentamicin and tobramicin. The concentration of the active agent is typically from about 0.1% to about 50% by weight of the gel composition, preferably from about 0.1% to about 20% by weight of the composition, most preferably from about 1% to about 20% by weight of the composition.

The incorporation of one or more therapeutic agents to the composition is optionally performed without solvents according to at least some embodiments of the present invention, by mixing the drug, preferably in the form of a fine powder, with the polymeric material by trituration. In this process, the dry small particle size powder, preferably in the range of from about 0.1 microns to about 20 microns, is mixed first with an equal amount of composition followed by mixing the obtained mixture with an equal amount of composition and so on, until a uniform composition is obtained.

Alternatively and according to at least some other embodiments of the present invention, the crosslinked gel is optionally immersed in concentrated drug solution prior to mixing in branched polymers; after absorption, the particles are preferably isolated prior to mixing in the continuous branched or linear polymer to form a drug loaded injectable gel. Without wishing to be limited by a single hypothesis, it is possible that for this preparation, a low burst type of release for the drug would be expected and the release would be expected to be slower as it is affected by the particle loading.

According to at least some embodiments of the present invention, the drugs comprise one or more anticancer drugs such as paclitaxel and cisplatin that can be injected into the tumor for localized regional drug therapy. Similarly, bupivacaine may optionally be loaded in this gel and the composition loaded in a ready to use formulation in a syringe for localized controlled drug delivery. Also, antibiotic drugs for treating infections may optionally be prepared by mixing gentamycin in the polymer paste prior to apply. Protein drugs that are preferably delivered systemically are also optionally loaded in the polymer paste and injected in the body for an extended release profile.

The gel composition can also contain radiopaque agents in order to track the performance of application and to instantaneously detect potential leakage. The radiopaque agents can be of organic or inorganic nature such as barium sulfate (BaSO4) zirconium oxide (ZrO₂).

In another preferred embodiment, radiopaque particles with an average diameter of about 250 to 600 μm, preferably 500 μm are added to the biomaterial. A preferred particle material is gold or titanium.

The gel compositions may also optionally contain one or more pharmaceutically acceptable additives or excipients. The additives may modify or affect one or more of the physical and/or mechanical properties of the polymer compositions. For example, the polymer compositions may contain nanoparticles and microparticles prepared from or containing biodegradable polymers, ceramics, absorbable inorganics, and combinations thereof for better control of tissue filling and duration.

The concentration of the additives and excipients is typically from about 0.01% to about 60% by weight of the gel composition, preferably from about 0.1% to about 30% by weight of the compositions, more preferably from about 0.1% to about 10% by weight of the composition.

### Methods of Administration of the Polymer gel

A polymer gel according to various embodiments of the present invention can be administered to a subject in a number of ways, which are well known in the art. Hereinafter, the term “subject” refers to the human or lower animal to whom the gel was administered. Preferably, administration occurs through injection or insertion. Injection may optionally be performed with a needle and syringe, while insertion is optionally performed with a catheter.

“Needle”, as used herein, refers to devices that can be used to administer, deliver, inject, or otherwise introduce the gel compositions to a subject for tissue repair and/or augmentation. Thus, as defined herein, needle includes needle, all needle-like devices, and all other annular introduction devices, such as tubing, etc. Specific examples include needles, hypodermic needles, surgical needles, infusion needles, catheters, trocars, cannulas, tubes, and tubing used for clinical, surgical, medical, procedural, or medical purposes. In one embodiment, the gel composition is administered by injection, for example, via a syringe. For deep implantation in the body, the syringe may be connected to a tube or catheter fitted to the outlet for administering the liquid polymers into a site within the body. An automated injector may be used for better control of the injection of the gel composition.

Another optional method for administration is in conjunction with a medical device. According to at least some embodiments of the present invention, the gel composition is used for coating a medical device or used in conjunction with a medical device for implantation and/or bone fill material. Such a method of administration may optionally be used (as a non-limiting list only) to enhance osseous integration, control hemostasis, control pain, provide anti-microbial factors to prevent infection, and/or to provide anti-tumor factors.

### Degradable and Non-Degradable Applications

According to at least some embodiments, the gel composition is provided in environmentally degradable and/or biodegradable formats, which may optionally have controlled rates of degradation that are quite slow. Non-limiting examples of applications of such material include biomedical, pharmaceutical, agricultural, and packaging applications.

Applications may also optionally include bioplastics used for disposable items, such as packaging and catering items (crockery, cutlery, pots, bowls, straws, organic waste bags, where they can be composted together with the food or green waste. Some trays and containers for fruit, vegetables, eggs and meat, bottles for soft drinks and dairy products and blister foils for fruit and vegetables are manufactured from bioplastics.

Non-disposable applications, which preferably have slower rates of environmental degradation, include but are not limited to mobile phone casings, carpet fibres, and car interiors, fuel line and plastic pipe applications, and new electroactive bioplastics are being developed that can be used to carry electrical current. In these areas, the goal is not biodegradability, but to create items from sustainable resources.

According to at least some embodiments, there is provided a gel composition for use with pressure control sensors, and/or for biodegradable sensors and biological sensors.

### EXAMPLES

The present invention may be more readily understood with reference to the following illustrative examples.

The following specific examples illustrate various aspects of the present invention, and are not intending to be limiting in any way.

### Example 1

The below non-limiting Example relates to a composition comprising cross-linked castor oil and branched castor oil (both prepared with citric acid).

### Cross Linked Polymer Synthesis:

92.5 g Castor oil and 7.5 g citric acid were added to a round-bottom flask equipped with a hermetic stopper, an inlet and outlet adapters for purging gas, and a magnetic stirrer. The reaction mixture was heated to 155° C. under a flow of nitrogen gas while being stirred magnetically until a clear homogenous melt was observed. The obtained pre-polymer was post-polymerized under vacuum (˜8 mbar) at 155° C. temperature until reaching the gelation point at which the reaction melt became elastomeric and the magnetic stirrer stopped its rotation. Then, the cross linked polymer was transferred into a flat glass pot and the polymer was cured at 140° C., 25 mbar for 2 days in a vacuum oven.

### Branched Polymer Synthesis:

93.5 g Castor Oil and 6.5 g citric acid were added to a round-bottom flask equipped with a hermetic stopper, an inlet and outlet adapters for purging gas, and a magnetic stirrer. The reaction mixture was heated to 155° C. under a flow of nitrogen gas while being stirred magnetically until a clear homogenous melt was observed. The obtained pre-polymer was post-polymerized under vacuum (˜8 mbar) for 3 days 155° C. The reaction progress was monitored by Gel Permeation Chromatography (GPC) and the reaction was stopped when the molecular weight stayed constant.

### Formulation Preparation

The composition was prepared by mixing the branched polymer and cross linked polymer as follows:

The cross link polymer was ground (milled) in liquid nitrogen and sieved through 10-20 mesh sieves while still frozen. The obtained particles of cross linked polymer were mixed with the branched polymer at 33:67% w/w ratio in a round-bottom flask at 120° C. applying constant stiffing for 2 hours under nitrogen atmosphere. Then, the resulting gel was removed from heating device and allowed to cool down to room temperature under nitrogen atmosphere. Finally, the prepared formulation gel was filled into a jar at aseptic conditions and stored in a closed container protected from light.

### Example 2

This Example relates to preparation of a composition featuring a polyester.

### Cross Linked Polymer Synthesis:

Castor oil and 7.5% w/w citric acid were inserted into a flask with a magnetic stirrer. The reaction was stirred in a nitrogen atmosphere at a temperature that was at least the acid melting point temperature (for this example 155° C. was used) until a homogenous solution was observed. After the solid acid was melted, the nitrogen was removed and the reaction continued under vacuum for three days, until the liquid become elastomeric. Then the crossed linked polymer was transferred into a flat glass pot and placed in a vacuum oven at 140° C., 25 mbar, for two days.

### Branched Polymer Synthesis:

This Example describes a preferred but illustrative branched component according to at least some embodiments of the present invention and an exemplary method of preparation thereof. The description relates to a branched component with polyester chains of hydroxy acids, as well as to a branched component without polyester chains of hydroxy acids.

Branched Polymer without Polyester Chains of Hydroxy Acids:

Castor oil and 6.5% w/w of citric acid were inserted into a flask with stiffing by a magnetic stirrer. The reaction was stirred in a nitrogen atmosphere at acid melting point temperature (155° C. for citric acid) until a homogenous solution was observed. After the solid acid was dissolved, the nitrogen was removed and the reaction continued in vacuum for 3 days. This reaction was monitored by GPC with specified and standardized molecular weight standards, and the reaction was deemed complete and stopped when the molecular weight of the resultant branched product remained constant.

Branched Polymer with Polyester Chains of Hydroxy Acids:

Addition of the polyester to the branched polymer was achieved by ring opening polymerization (ROP) of caprolactone on the branched polymer. The ROP occurred in the presence of a catalyst (Zn-lactide), added in amount 0.1% mole per mole caprolactone. Caprolactone was used in the amount of 40% w/w of weight of the branched polymer. The caprolactone, branched polymer and the catalyst were inserted into a flask with a magnetic stirrer. The reaction was stirred in an argon atmosphere at 140° C. for 3 days. The reaction was monitored by GPC and the reaction was deemed complete and stopped when the molecular weight remained constant.

### Preparation of the Mixture

This Example describes a preferred but illustrative mixture according to at least some embodiments of the present invention and an exemplary method of preparation thereof.

The polymer gel comprised a mixture of the crosslinked polymer and the branched polymer; in this Example, the branched polymer featured polyester chains.

The crosslinked polymer component was prepared by milling in liquid nitrogen, and was then sieved in a 15 mesh sieve. The resulting milled crosslinked polymer component was added to a flask with the branched polymer which is liquid. The crosslinked polymer component was 33% of the total mass. The mixture was stirred at 120° C. for two hours under nitrogen. The resulting mixture was then allowed to cool back to room temperature in a nitrogen atmosphere. The polymer gel was thus ready to be administered, for example by being inserted into syringes for injection.

### Example 3

### In Vivo Testing of the Gel

This Example describes testing of compositions according to at least some embodiments of the present invention, referred to herein as a “gel” as the composition is preferably in that form for the uses described herein. The study evaluated the persistence (ie stability and maintenance at a particular tissue location) and tissue biocompatibility of the compositions.

Methods

Animals: 30 Inbred Sprague-Dawley (SD) female rats were used (Harlan Laboratories Ltd, Ein Kerem Breeding Farm, Jerusalem). The age/weight range at start of study was 200-240 grams. The animals were healthy and were not pregnant or lactating.

Before the study began, the animals were acclimatized for 4-5 days. They were maintained under pathogen free conditions under standard light/darkness 12 hours cycling regimen. Food (rodent chow) and water were given ad libitum.

For administration of the composition according to the present invention, the animals were first anesthetized with 85% Ketamine HCl (Ketaset™, 100 mg/mL, Fort Dodge)/15% Xylazine HCl (20 mg/mL, Biob, France). The administered dose of the composition was 120 μL/100 g body weight; it was given i.p.

Upon completion of the experiment, the animals were euthanized with pentobarbitone sodium 200 mg/mL (Pental, CTS, Israel).

The table below describes the tested compositions according to the present invention.

TABLE 1
description of the tested materials

| Test | Physical | Characterization/ | Storage |
|---|---|---|---|
| materials | description | Certification | conditions |
| DF-1 | Ready for | Control compound | At room |
| (MY040) | injection, as a | [castor oil:citric acid | temperature, |
| | oleaginous viscous | 93.5:6.5]: | protected |
| | pasty semisolid in | caprolactone | from light |
| | prefilled syringe | 60:40; branched only | |
| DF-2 | Ready for | Experimental | At room |
| (MY041) | injection, as a | Compound | temperature, |
| | oleaginous viscous | [Castor Oil:Citric | protected |
| | gel in prefilled | Acid 93.5:6.5] + | from light |
| | syringe | [Castor Oil:Citric | |
| | | 92.5:7.5] | |
| | | 2:1 | |
| DF-3 | Ready for | Experimental | At room |
| (MY042) | injection, as a | Compound | temperature, |
| | oleaginous viscous | [Castor Oil:Citric | protected |
| | gel in prefilled | Acid 93.5:6.5]: | from light |
| | syringe | caprolactone | |
| | | 60:40 + | |
| | | [Castor Oil:Citric | |
| | | Acid 92.5:7.5] | |
| | | 2:1 | |
| DF-4 | Ready for | Control | At room |
| [Macrolane | injection, as a | Exp: January 2010 | temperature |
| VRF30_10 | hydrophilic | | |
| ml] | viscous gel in | | |
| | prefilled syringe | | |

DF-1 is an exemplary control composition, which is only branched. The abbreviated description of the composition is as follows: the composition features castor oil branched with citric acid in a ratio of 93.5% castor oil to 6.5% citric acid, weight per weight, with the addition of polyester chains of hydroxy acids. The branched material was reacted with caprolactone, in a ratio of 60% branched material to 40% caprolactone as described in Example 2 above. As described in greater detail below, the branched material alone was not persistent in the body and hence was not effective according to the various exemplary embodiments of applications described herein. The compositions according to various embodiments of the present invention that were effective featured both cross-linked and branched material mixed together.

DF-2 is an exemplary illustrative composition according to at least some embodiments of the present invention. The abbreviated description of the composition is as follows: the composition features a mixture of castor oil crosslinked with citric acid in a ratio of 92.5% castor oil to 7.5% citric acid, weight per weight; and castor oil branched with citric acid in a ratio of 93.5% castor oil to 6.5% citric acid, weight per weight; in a ratio of 2:1 branched material: cross-linked material, prepared as described in Example 1.

DF-3 is an exemplary illustrative composition according to at least some embodiments of the present invention. The abbreviated description of the composition is as follows: the composition features castor oil branched with polyester chains of hydroxy acids as described for DF-1, and castor oil crosslinked as described for DF-2. The ratio of branched material: cross-linked material was 2:1, prepared as described for preparation of the mixture in Example 2.

Macrolane™ VRF (referred to herein as “Macrolane”, manufactured by Q-Med, Sweden) is a soft tissue augmentation material, featuring stabilized hyaluronic acid, which is approved for use in humans in Europe, and was used as the control standard.

Experimental Protocol

Animals

Animals: 30 Inbred Sprague-Dawley (SD) female rats were used (Harlan Laboratories Ltd, Ein Kerem Breeding Farm, Jerusalem). The age/weight range at start of study was 200-240 grams. The animals were healthy and were not pregnant or lactating.

Before the study began, the animals were acclimatized for 4-5 days. They were maintained under pathogen free conditions under standard light/darkness 12 hours cycling regimen. Food (rodent chow) and water were given ad libitum.

For administration of the composition according to the present invention, the animals were first anesthetized with 85% Ketamine HCl (Ketaset™, 100 mg/mL, Fort Dodge)/15% Xylazine HCl (20 mg/mL, Biob, France). The administered dose of the composition was 120 μL/100 g body weight; it was given i.p.

Upon completion of the experiment, the animals were euthanized with pentobarbitone sodium 200 mg/mL (Pental, CTS, Israel).

The rats were divided randomly into 5 groups, each of which is described in a separate table below and an associated figure.

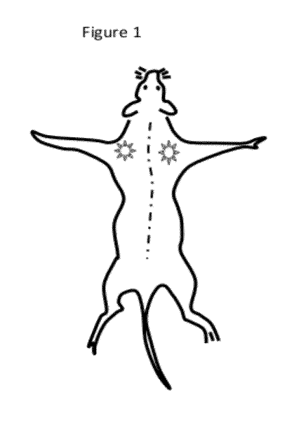

The description of experimental groups 1-4 is shown with regard to Table 2 and FIG. 1.

TABLE 2
experimental design groups 1-4

| | | Numbers of rats | Numbers of rats |
|---|---|---|---|
| | | tested after | tested after |
| | Materials | 1 month | 3 months |
| Group 1 | DF-1 | 3 | 3 |
| Group 2 | DF-2 | 3 | 3 |
| Group 3 | DF-3 | 3 | 3 |
| Group 4 | DF-4 | 3 | 3 |

Each rat in experimental groups 1-4 received two injections of 0.4 ml of the same material (FIG. 1 shows the injection locations for groups 1-4).

Group 5: three different compositions (DF) were injected into each rat as shown.

TABLE 3
experimental design group 5

| | | Numbers of rats | Numbers of rats |
|---|---|---|---|
| | | tested after | tested after |
| | Materials | 1 day | 3 days |
| Group 5 | DF-2, DF-3, | 2 | 2 |
| | DF-4 | | |

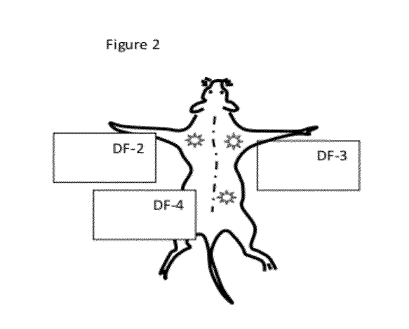

Each rat in experimental group 5 received three injections of 0.4 ml of DF 2-4 (see FIG. 2).

Results:

Implant persistence analysis included implant gross evaluation. The implant was carefully separated from the tissue. Its wet weight was determined, after which it was lyophilized for two days to determine dry weight.

DF-1 did not show persistence after one month, meaning that there was hardly any trace of this injected material in the three rats after one month, the experiment with this material was terminated and histopathological evaluations of implantation areas were not performed.

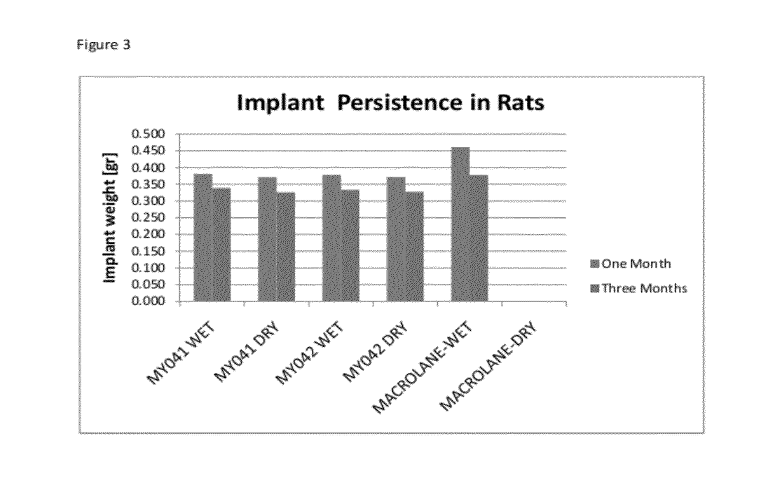

The implant weight (dry and wet) after one and three months is presented in FIG. 3. The results show that, as compared to the standard composition in the art, the tested compositions of the present invention showed greater persistence and lower weight reduction.

Therefore, DF-1, featuring only the branched material alone, was not persistent in the body and hence was not effective according to the various exemplary embodiments of applications described herein. The compositions according to various embodiments of the present invention that were effective featured both cross-linked and branched material mixed together, as described with regard to the various illustrative compositions according to various embodiments of the present invention.

Weight Loss:

This particular experiment was designed to evaluate biocompatibility, injectability and grossly estimate the implant persistence. The weight analysis revealed that DF-2 and DF-3 lost around 10% of weight (dry and wet) between the one month and three month test points (post implantation). In the same period of time Macrolane showed weight loss of about 18%. Macrolane weights were higher both at one month and at three months, although the initial injected volume was similar, probably due to the fact that Macrolane density is higher than the tested compositions according to at least some embodiments of the present invention.

These results correlate well with initial data from persistence experiment as presented in table 4.

TABLE 4
Implant amount [%] remained after one
month subcutaneous implantation in rats

| Material | Remaining implant amount [%] |
|---|---|
| DF-3-DRY | 98.1 ± 6.6 |
| DF-3-WET | 98.9 ± 6.8 |
| DF-2-DRY | 95.3 ± 2.3 |
| DF-2-WET | 97.3 ± 1.9 |

Remaining implant amount is expressed as % w/w of initially implanted amount.

Clinical Observations:

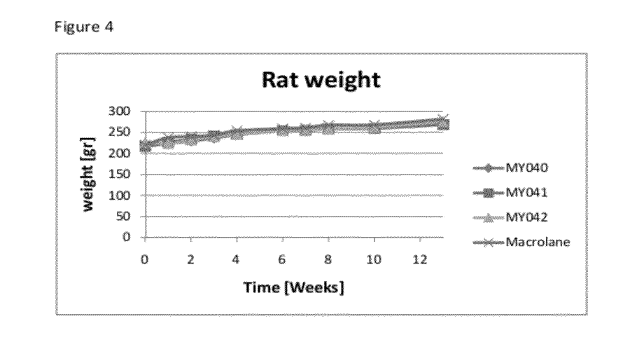

All animals survived the three months experiment and weight gain was similar to the control group (Macrolane) (FIG. 4). No signs of systemic toxicity were observed. In particular, no neurological deficiencies or behavioral changes, including signs of stress, were noted. The gross observation of the body cavities and organs did not reveal any polymer-related lesions and abnormalities.

Histopathological Evaluation:

Objective of the Study:

The study assessed the local tissue reaction following implantation, and compared the severity of the local reaction between the different implant materials.

### Organ/Tissue Collection & Fixations:

Tissues were collected during the respective scheduled necropsy sessions and fixed in 10% neutral buffered formalin (approximately 4% formaldehyde solution) for at least 48-hr fixation period prior to their shipment to Patholab, Israel.

### Slides Preparation & Histopathological Examinations:

Slide preparation was done at Patholab. Tissues were trimmed, embedded in paraffin, sectioned at approximately 5 microns thickness and stained with Hematoxylin & Eosin (H&E). Histological evaluation was done by Dr. Abraham Nyska, D.V.M., Dipl. ECVP, Expert in Toxicologic Pathology.
The evaluation of histopathological changes was based on the following scoring system:

TABLE 5
scoring system

| CELL TYPE/ | SCORE | | | | |
|---|---|---|---|---|---|
| RESPONSE | 0 | 1 | 2 | 3 | 4 |
| Polymorphonuclear | None | Rare | Slight | Heavy | Packed |
| cells | | 1-5/phf | 5-10/phf | Infiltrate | |
| Eosinophils | | | | | |
| Lymphocytes | | | | | |
| Plasma cells | | | | | |
| Macrophages | | | | | |
| Giant cells | | | | | |
| Necrosis | None | Minimal | Mild | Moderate | Severe |
| Fibroplasia | None | Minimal capillary | Groups of 4-7 | Broad band of | Extensive band of |
| | | proliferation | capillaries with | capillaries with | capillaries with |
| | | focal, 1-3 buds | supporting fibroblastic | supporting Structures | supporting fibroblastic |
| | | | structures | | structures |
| Fibrosis | None | Narrow band | Moderately thick band | Thick band | Extensive band |
| Edema | None | Narrow band | Moderately thick band | Thick band | Extensive band |

### The Criteria for Assessment of Tolerability was as Follows:

When the inflammatory cell infiltration was severe (i.e., extensive, packed—grade 4), the tolerability was assessed as low.
When the inflammatory cell infiltration was moderate (i.e., heavy, thick, grade 3), the tolerability was assessed as medium.
When the inflammatory cell infiltration was mild (grade 2), the tolerability was assessed as good.
When the inflammatory cell infiltration was minimal (grade 1), the tolerability was assessed as excellent.

### Histopathological Findings and Assessment:

DF-2: There was a capsular reaction formation surrounding the cavity, with no evidence for presence of the experimental composition in any of the sections. The initial (1-day) inflammatory reaction only partially subsided within 1-month, with evidence of mild histiocytic and lymphocytic reaction. The tolerability within a month is considered as good. Within 3-month the subcutaneous implantation reaction consisted of a highly mature, fibrotic capsular reaction surrounding a cavity. There was evidence for on-going inflammatory reaction, consisting of grade 1 to 2 layer of macrophages, grade 1 to 2 of polymorphonuclear cells, with minimal to mild (grade 1 to 2) presence of mononuclear cells, and minimal (grade 1) presence multinucleated giant cells.

DF-3: Capsular reaction formation surrounding the cavity was found, with no evidence for presence of the filler in any of the sections. The initial (1-day) inflammatory reaction only partially subsided within 1-month, with evidence of mild histiocytic and minimal multinucleated giant cells reaction. There was a time-related increase (grade 1-2) in the mononuclear (lymphocytic) reaction. The tolerability within a month is considered as good. Within 3-month the subcutaneous implantation reaction consisted of a highly mature, fibrotic capsular reaction surrounding a cavity. There was an on-going inflammatory reaction, consisting of grade 1 to 2 layer of macrophages, with minimal to mild (grade 1 to 2) presence of mononuclear cells, and minimal (grade 1) presence multinucleated giant cells and eosinophils.

DF-4 (MACROLANE): This substance showed similar effects to the above materials in terms of the response of the animals to the implanted material. There is capsular reaction formation surrounding the cavity containing the filler (present in all sections). The initial (1-day) inflammatory reaction subsided completely within 1-month, with no evidence of multinucleated giant cells reaction. The tolerability within a month is considered as excellent. Within 3-month the subcutaneous implantation reaction consisted of a highly mature, fibrotic capsular reaction surrounding a cavity. There was no evidence for on-going inflammatory reaction. No multinucleated giant cell reaction was noted.

### Example 4

This Example describes a preferred but illustrative gel according to at least some embodiments of the present invention and an exemplary method of preparation thereof. Specific examples of materials prepared according to these parameters are described in greater detail below.

### General Materials and Synthesis

### Cross Linked Polymer Synthesis:

90-92.5% Castor oil and 7.5%-10% w/w citric acid are inserted into a flask with a magnetic stirrer. The reaction is stirred in a nitrogen atmosphere at a temperature that was at least the acid melting point temperature (130-155° C.) until a homogenous solution is observed. After the solid acid is melted, the nitrogen is removed and the reaction continues under vacuum for one to ten days, until the liquid become elastomeric. Then the crossed linked polymer is transferred into a flat glass pot and placed in a vacuum oven at 120-160° C., 5-30 mbar for one to four days.

### Branched Polymer Synthesis:

This Example describes a preferred but illustrative branched component according to at least some embodiments of the present invention and an exemplary method of preparation thereof. The description relates to a branched component with polyester chains of hydroxy acids, as well as to a branched component without polyester chains of hydroxy acids.

Branched Polymer without Polyester Chains of Hydroxy Acids:

93-96% w/w Castor oil and 4-7% w/w Citric Acid or Sebasic Acid are inserted into a flask with stirring by a magnetic stirrer. The reaction is stirred in a nitrogen atmosphere at acid melting point temperature (155° C. for citric acid or 180° C. for sebasic acid until a homogenous solution is observed. After the solid acid is dissolved, the nitrogen is removed and the reaction continues in vacuum for one to five days. This reaction is monitored by GPC (gel permeation chromatography) with specified and standardized molecular weight standards, and the reaction is deemed complete and stopped when the molecular weight of the resultant branched product remains constant.

Branched Polymer with Polyester Chains of Hydroxy Acids:

Addition of the polyester to the branched polymer is achieved by ring opening polymerization (ROP) of caprolactone or lactide on the branched polymer. The ROP occurs in the presence of a catalyst Zn-lactide, added in amount 0.1% mole per mole caprolactone or lactide. Caprolactone is used in the amount of 40% w/w of weight of the branched polymer. The caprolactone, or lactide, branched polymer and the catalyst are inserted into a flask with a magnetic stirrer. The reaction is stirred in an argon atmosphere at 140° C. for 2-4 days. The reaction is monitored by GPC and the reaction is deemed complete and stopped when the molecular weight remains constant.

### Preparation of the Mixture

This Example describes a preferred but illustrative mixture according to at least some embodiments of the present invention and an exemplary method of preparation thereof.

The polymer gel comprised a mixture of the crosslinked polymer and the branched polymer the branched polymer component may optionally feature a polyester or may not feature the polyester as described above).

The crosslinked polymer component is prepared by milling in liquid nitrogen, and is sieved in a 10-20 mesh sieve. The resulting milled crosslinked polymer component is added to a flask with the branched polymer which is liquid. The crosslinked polymer component is between 10% to 50 w/w of the total mass. The mixture is stirred at 100-150° C. 120° C. for one to ten hours under nitrogen. The resulting gel is then allowed to cool back to room temperature in a nitrogen atmosphere. The polymer gel is thus ready for use.

The gel preferably has a viscosity in the range of about 10⁵-10⁶ cP units (at a low shear rate) depending on the ratio of hydroxy acid monomer and castor oil and on the molecular weight of the hydroxy acid chains; as the hydroxy acid chain length increases, the viscosity increases.

### Specific Examples of Materials

The following materials were synthesized as described above. The materials used are listed below.

TABLE 6
Crosslinked Polymer

| | Castor oil % | Citric acid % |
|---|---|---|
| Code | w/w | w/w |
| MY013 | 93 | 7 |
| MY023 | 92.5 | 7.5 |
| MY043 | 92.5 | 7.5 |
| BV 015 | 85 | 15 |
| BV016 | 90 | 10 |
| BV017 | 92.5 | 7.5 |

TABLE 7
Branched Polymer

| | Castor oil % | Citric acid % | |
|---|---|---|---|
| Code | w/w | w/w | Caprolactone |
| MY021 | 94.5 | 5.5 | — |
| MY022 | 93.5 | 6.5 | — |
| MY040 | 56.1 | 3.9 | 40 |

TABLE 8
Gel

| | Cross linked | | Branched | |
|---|---|---|---|---|
| Code | polymer Type | % w/w | Polymer Type | % w/w |
| MY044 | MY023 | 33 | MY040 | 66 |
| MY045 | MY023 | 33 | MY022 | 66 |
| MY046 | MY023 | 33 | MY021 | 66 |
| MY047 | MY023 | 20 | MY040 | 80 |
| MY048 | MY023 | 20 | MY022 | 80 |
| MY049 | MY043 | 20 | MY021 | 80 |
| MY050 | MY023 | 10 | MY040 | 90 |
| MY051 | MY043 | 10 | MY022 | 90 |
| MY052 | MY043 | 10 | MY021 | 90 |

### Example 5

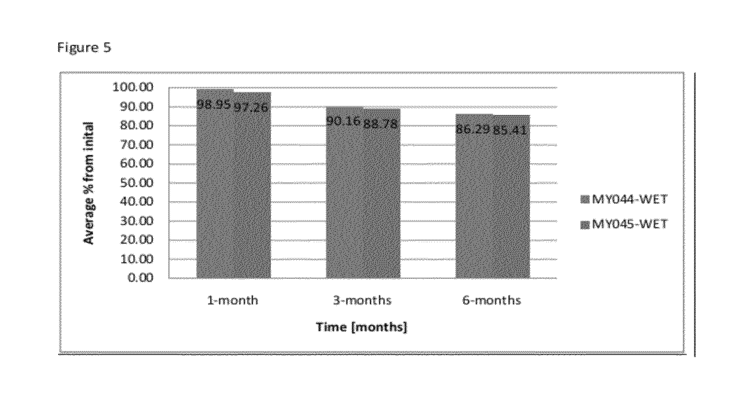

This Example describes testing of compositions MY044 and MY045 from example 4. The study evaluated the persistence (i.e. stability and maintenance at a particular tissue location) and tissue biocompatibility of the compositions after six months from implant according to the same methods detailed in example 3. The persistence results are summarized in table 9 and FIG. 5.

TABLE 9
Implant amount [%] remained after 1, 3,
6 months subcutaneous implantation in rats

| | MY044- | MY044- | MY045- | MY045- |
|---|---|---|---|---|
| | DRY | WET | DRY | WET |
| | Implant | Implant | Implant | Implant |
| | weight | weight | weight | weight |
| | change | change | change | change |
| | % | % | % | % |
| 1-month | 98.11 ± 6.6 | 98.95 ± 6.8 | 95.27 ± 2.3 | 97.26 ± 1.9 |
| 3-months | 88.89 ± 1.7 | 90.16 ± 1.7 | 86.97 ± 4.3 | 88.78 ± 4.2 |
| 6-months | 84.03 ± 2.9 | 86.29 ± 3.4 | 82.60 ± 1.5 | 85.41 ± 2 |

### Histopathological Findings and Assessment:

The individual findings are presented in the table as follows. Samples from each group were photographed.

TABLE 10
histopathological findings

| Filler number | MY044 | | | | MY045 | | | |
|---|---|---|---|---|---|---|---|---|
| Patho-lab serial num | 1 | 2 | 3 | 4 | 5 | 6 | 7 | 8 |
| Implement Number | 1 | | | | 1 | | | |
| | | 9 | 14 | 5 | | 8 | 5 | 13 |
| | 2 | | | | 7 | | | |
| Finding | | | | | | | | |
| Polymorphonuclear cells | | | | | | | | |
| Eosinophils | | | | | | | | |
| Lymphocytes | 1 | 0-1 | 1 | 0-1 | 1 | 1 | 0-1 | 0-1 |
| Plasma cells | | | | | | | | |
| Macrophages | | | | | | | | |
| Giant cells | | | | | | | | |
| Necrosis | | | | | | | | |
| Fibroplasia | | | | | | | | |
| Fibrosis | 1 | 1 | 1 | 1 | 1 | 1 | 1 | 1 |
| Edema | | | | | | | | |
| X—NO IMPLANTATION | | | | | | | | |
| SITE IS PRESENT IN | | | | | | | | |
| SECTION | | | | | | | | |

### MY044:

### 6-Month Evaluation (Samples 5, 9, 12, 14)

The subcutaneous implantation reaction consisted of a highly mature, fibrotic capsular reaction surrounding a cavity. There was no to minimal (grade 0 to 1) evidence for mononuclear lymphocytic infiltration.

No multinucleated giant cells reaction was noted.

### General Grading of Grade of Tolerability—Excellent

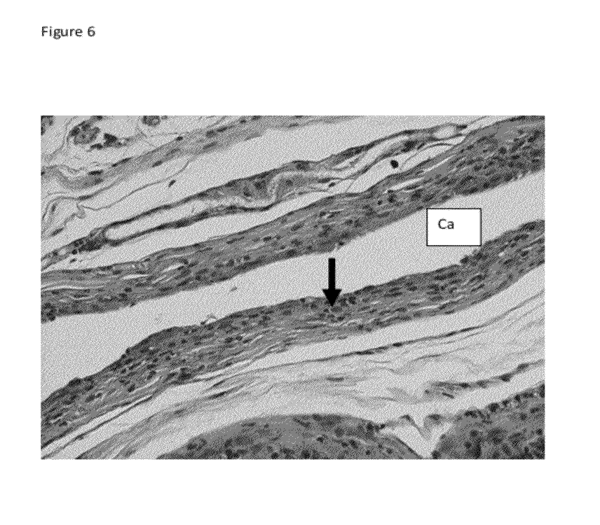

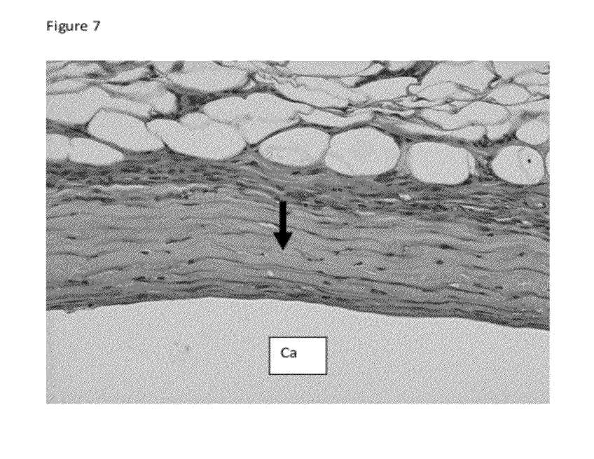

Typical histopathological reactions are shown in the photographs of FIGS. 6-7 and are further described as follows:

### Sample 12: (Results Shown in FIG. 6)

This photograph features ×20 magnification view of the capsular reaction:

### Ca-cavity

There is excellent, time-related progressive maturation of the capsule (arrow), with minimal (grade 1) evidence for mononuclear cell infiltration

### Sample 9: (Results Shown in FIG. 7)

This photograph features ×20 magnification view of the capsular reaction:

### Ca-cavity

There is excellent, time-related progressive maturation of the capsule (arrow), with no (grade 0) evidence for mononuclear cell infiltration

### Filler MY045:

### 6-Month Evaluation (Samples 5, 8, 13, 17):

The subcutaneous implantation reaction consisted of a highly mature, fibrotic capsular reaction surrounding a cavity. There was no to minimal (grade 0 to 1) evidence for mononuclear lymphocytic infiltration.
No multinucleated giant cells reaction was noted.

### General Grading of Grade of Tolerability—Excellent

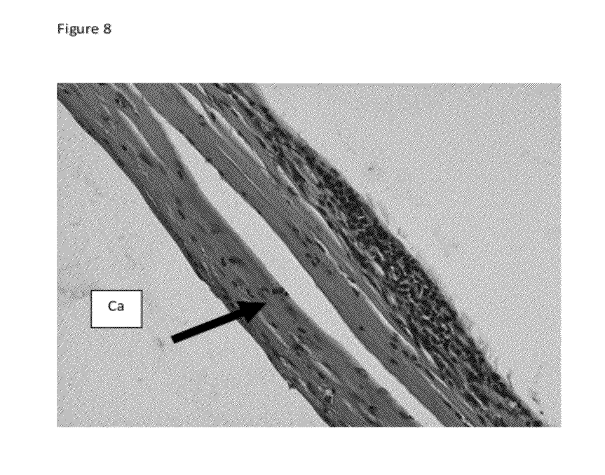

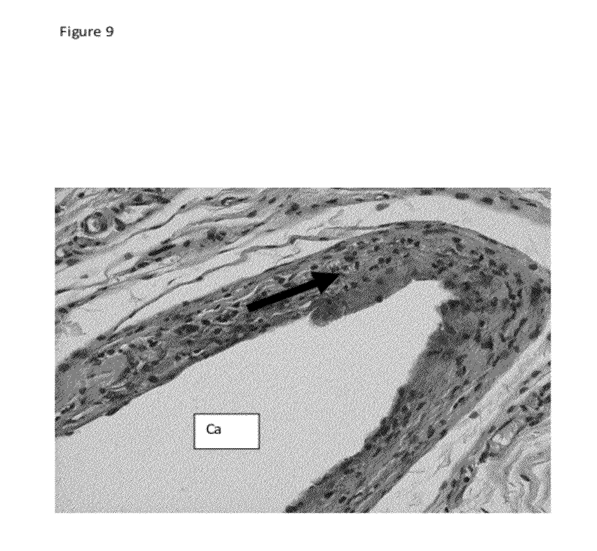

Typical histopathological reactions are shown in the photographs of FIGS. 8-9 and are further described as follows

### Sample 17: (Results Shown in FIG. 8)

This photograph features ×20 magnification view of the capsular reaction:

### Ca-cavity

There is excellent, time-related progressive maturation of the capsule (arrow), with grade 1 evidence for mononuclear cell infiltration

### Sample 8: (Results Shown in FIG. 9)

This photograph features ×20 magnification view of the capsular reaction:

### Ca-cavity

There is excellent, time-related progressive maturation of the capsule (arrow), with grade 1 evidence for mononuclear cell infiltration

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A biologically compatible gel comprising at least one cross-linked polymer and at least one branched polymer, wherein each of said cross-linked polymer and said branched polymer comprises one or more of castor oil, ricinoleic acid and/or hydroxyl stearic acid as a base.

2. The gel of claim 1, wherein said branched polymer comprises a polyester.

3. The gel of claim 1, wherein said cross-linked material comprises a cross-linker, comprising an acid containing two or more carboxylic or alcohol groups.

4. The gel of claim 3, wherein said acid comprises one or more of citric acid, mucic acid, tartaric acid, sebacic acid or succinic acid or a combination thereof, in an amount suitable to induce cross-linking.

5-6. (canceled)

7. The gel of claim 4 wherein said cross-linker comprises citric acid in an amount of at least 7% w/w with regard to the base.

8. The gel of claim 7, wherein said citric acid as a cross-linker is present in an amount of from about 7% to about 20%.

9-13. (canceled)

14. The gel of claim 2, wherein said branched polymer comprises a branching agent.

15. The gel of claim 14, wherein said branching comprises an acid containing two or more carboxylic or alcohol groups.

16. The gel of claim 15, wherein said acid comprises one or more of citric acid, mucic acid, tartaric acid, sebacic acid or succinic acid or a combination thereof, in an amount suitable to induce branching.

17. The gel of claim 16, wherein said acid is present in an amount of from about 0.1% to about 7% as a branching agent.

18. (canceled)

19. The gel of claim 16, wherein said base comprises castor oil.

20-21. (canceled)

22. The gel of claim 19, wherein said polyester is added through reaction with a lactone.

23. The gel of claim 22, wherein said lactone comprises caprolactone and/or lactide.

24. The gel of claim 19, wherein said polyester is added through direct condensation with the hydroxy acid.

25. The gel of claim 2, wherein said cross-linked polymer and said branched polymer are mixed to form said gel.

26. The gel of claim 2, wherein said cross-linked polymer is present in an amount of from 10% to 50% of the gel, weight per weight percent.

27. The gel of claim 1, adapted for having a controlled rate of biodegradation.

28. A method of treatment, comprising placing the gel of claim 1 into a body of a subject for volumetrically filling any void, for augmenting any soft tissue or for a combination thereof.

29. (canceled)

30. The method of claim 28, for performing a function selected from the group consisting of smoothing (filling) nasolabial folds, enhancing cheekbones, augmenting lips, smoothing (filling) mentolabial folds, enhancing the chin, enhancing the bridge of the nose, camouflaging scars, filling depressions, smoothing out irregularity, correcting asymmetry in facial hemiatrophy, second bronchial arch syndrome, facial lipodystrophy, providing the feel of natural fat when implanted in the body and camouflaging age-related wrinkles or a combination thereof.

31. The method of claim 28, for performing a function, selected from the group consisting of urethral bulking agent for the treatment of female urinary incontinence, intra-articular injection, restoring or improving sphincter function, treatment of vesicoureteral reflux, treating stress urinary incontinence, periurethral injection, ureteral injection, injection into the tissues of the gastrointestinal tract for the bulking of tissue to prevent reflux; to aid in sphincter muscle coaptation, internal or external, and for coaptation of an enlarged lumen; intraocular injection for the replacement of vitreous fluid or maintenance of intraocular pressure for retinal detachment; injection into anatomical ducts to temporarily plug the outlet to prevent reflux or infection propagation; injection to the joints for managing Osteoarthritis and larynx rehabilitation after surgery or atrophy.

32-35. (canceled)

36. A method of preparing the gel of claim 1, comprising separately preparing the branched and cross-linked polymers; mixing said branched and cross-linked polymers to form a mixture; and curing said mixture through a vacuum oven without any added solvents.

37. The method of claim 36, wherein said preparing each of the branched polymer and the cross-linked polymer comprises mixing said base with an acid as said branching or cross-linking agent, respectively to form a liquid; and continuing the reaction under vacuum until said liquid becomes elastomeric.

38. The method of claim 37, wherein said acid comprises a solid acid and wherein said mixing said base with said acid is performed at a temperature that is at least a melting temperature of said solid acid.

39. The method of claim 38, wherein said preparing said cross-linked polymer further comprises forming a powder of said cross-linked polymer; and wherein said preparing said branched polymer further comprises forming a liquid or paste of said branched polymer.

40. The method of claim 39, further comprising subjecting said mixture to a process comprising one or more of extrusion, injection, compression molding, particulate leaching or solvent casting.

41-43. (canceled)
